(19) United States
(12) Patent Application Publication
(10) Pub. No.: US 2017/0348122 A1
(43) Pub. Date: Dec. 7, 2017

(54) EXPANDABLE STENT HAVING A CONSTANT LENGTH

(71) Applicant: Contego Medical, LLC, Raleigh, NC (US)

(72) Inventors: Ravish Sachar, Raleigh, NC (US); Tanhum Feld, Moshav Merhavya (IL)

(21) Appl. No.: 15/411,437

(22) Filed: Jan. 20, 2017

Related U.S. Application Data
(63) Continuation of application No. 14/213,668, filed on Mar. 14, 2014, now abandoned.
(60) Provisional application No. 61/794,924, filed on Mar. 15, 2013.

(51) Int. Cl. A61F 2/915

(52) U.S. Cl. CPC A61F 2/915; A61F 2002/91533; A61F 2002/91558

(57) ABSTRACT

Implementations described herein comprise expandable stents configured to maintain a substantially constant length between a collapsed position and a deployed position having a plurality of spring members and a plurality of biasable couplings connecting adjacent spring members. Each spring member includes a plurality of integrally connected wave members formed in a ring shape, each wave member has a plurality of curved segments having a plurality of substantially straight segments disposed therebetween. The plurality of biasable couplings are adapted to elongate upon movement of the stent from the collapsed position to the deployed position to proportionally compensate for the decrease in amplitude of the plurality of wave members of the plurality of spring members such that the stent maintains a substantially constant length.

### CROSS REFERENCE TO RELATED APPLICATION

This application is a continuation of U.S. patent application Ser. No. 14/213,668, filed Mar. 14, 2014, which claims the benefit of the U.S. Provisional Application No. 61/794,924, filed Mar. 15, 2013. Each of these applications are incorporated by reference in their entireties for all purposes.

### BACKGROUND

### Field of the Invention

The present invention relates generally to stents and, more particularly, to stents that maintain a substantially constant length when moved from a collapsed position to a deployed position.

### Related Art

Stents are commonly used to treat vessel diseases such as stenoses, strictures, thrombosis, and aneurysms. Stents can be configured to perform a variety of different functions such as, for example and without limitation, reinforcing vessel walls and increasing cross sectional area and, thus, volumetric flow. Typically, stents are employed to restore or maintain healthy blood flow.

Various types of stent architectures are known in the art. One shortcoming of conventional stents is that they may have deficiencies described collectively as “edge effects.” In one aspect, the overall length of the stent shortens as the stent is moved from a collapsed configuration to a deployed configuration. Edge effects due to stent shortening can lead to unpredictability of stent placement. In aspects where a balloon is used to deploy the stent, if the overall length of the stent decreases during deployment and the balloon is exposed, the balloon can over expand at one or both end of the stent. Edge effects due to balloon over expansion can lead to injury of the vessel wall.

Accordingly, a need exists for stents configured to maintain a substantially constant length between a collapsed and a deployed configuration.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Aspects of the present invention comprise an expandable stent for placement in a blood vessel comprising a plurality of spring members and a plurality of biasable couplings connecting adjacent spring members. Here, the plurality of spring members defines a stent having a longitudinal axis, wherein the plurality of spring members can be configured to move between a collapsed position and a deployed position. Each spring member can comprise a plurality of integrally connected wave members formed in a ring shape that can be positioned in a ring plane substantially perpendicular to the longitudinal axis. Further, each wave member can comprise a plurality of curved segments having a plurality of substantially straight segments disposed therebetween. In other aspects, the plurality of biasable couplings have opposing ends 122, 124, wherein one end of one biasable coupling can be joined to a portion of the respective curved segment of a select wave member of a selected spring member and the other opposing end of the one biasable coupling can be joined to a portion of the respective curved segment of another select wave member of an adjacent spring member. It is contemplated that the plurality of biasable couplings can be adapted to elongate upon movement of the stent from the collapsed position to the deployed position to proportionally compensate for the decrease in amplitude of the plurality of wave members of the plurality of spring members such that the stent maintains a substantially constant length.

Additional features and advantages of exemplary aspects of the disclosure will be set forth in the description which follows, and in part will be obvious from the description, or may be learned by the practice of such exemplary aspects. The features and advantages of such aspects may be realized and obtained by means of the instruments and combinations particularly pointed out in the appended claims. These and other features will become more fully apparent from the following description and appended claims, or may be learned by the practice of such exemplary aspects as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates one implementation of a stent of the present disclosure, where the stent has been cut longitudinally and laid flat.

FIG. 2 illustrates a detailed view of one implementation of a biasable coupling of the present disclosure.

FIG. 3 illustrates a perspective view of one implementation of a stent of the present disclosure.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more aspects of the invention. It is to be understood that the drawings are diagrammatic and schematic representations of one or more aspects, and are not limiting of the present disclosure. Moreover, while various drawings are provided at a scale that is considered functional for one or more aspects, the drawings are not necessarily drawn to scale for all contemplated aspects. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well known aspects of stent technology have not been described in particular detail in order to avoid unnecessarily obscuring aspects of the disclosed aspects.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

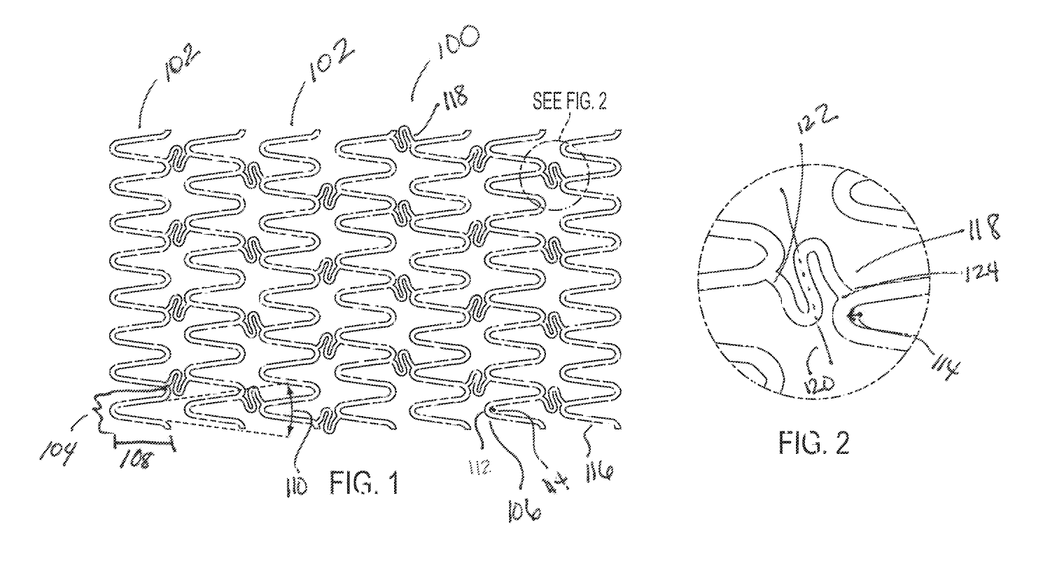

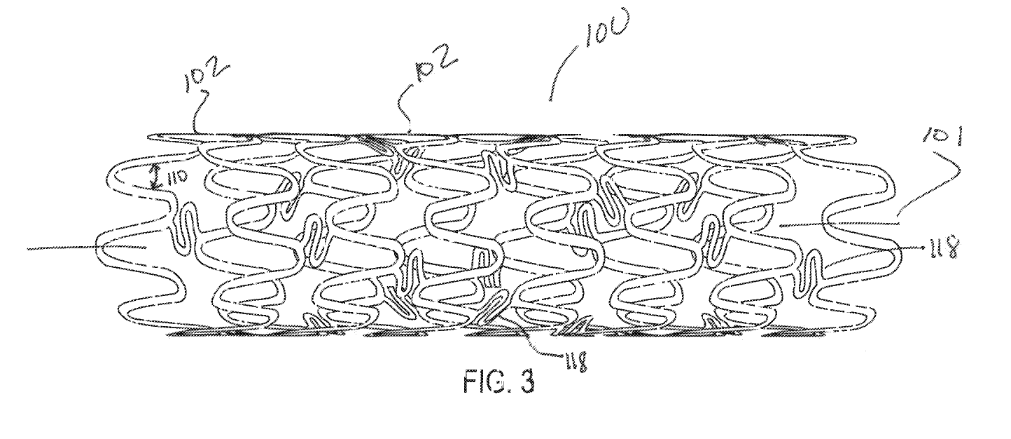

As shown in FIGS. 1-3, aspects of the present invention comprise an expandable stent 100 for placement in a blood vessel comprising a plurality of spring members 102 and a plurality of biasable couplings 118 connecting adjacent spring members, wherein the stent is adapted to maintain a substantially constant length when moved between a collapsed position and a deployed position. In certain aspects, the stent 100 can be adapted to be balloon-expandable. In certain other aspects, the stent can be adapted to have a deployed radial strength sufficient to overcome the recoil forces applied by the blood vessel and, optionally, the plaque layer, within which it is deployed. In certain other aspects, the stent is configured not to develop strains greater than about 45% in the stent material upon movement from the collapsed position to the deployed position. It is also contemplated that the stent 100 can be formed from conventional stent materials, for example and without limitation, stainless steel, Cobalt Chromium alloy and the like.

In certain aspects, the plurality of spring members 102 defines a stent 100 having a longitudinal axis 101, wherein the plurality of spring members can be configured to move between a collapsed position and a deployed position. It is contemplated that each spring member 102 can comprise a plurality of integrally connected wave members formed in a ring shape that can be positioned in a ring plane substantially perpendicular to the longitudinal axis of the stent 100. In other aspects, each wave member 104 can comprise a plurality of curved segments having a plurality of substantially straight segments 116 disposed therebetween.

In another aspect, the plurality of curved segments of each wave member 104 can have a radius 114. In certain aspects, the radius 114 can be from about 0.05 to about 0.15 mm and, more preferably, can be about 0.1 mm. In other aspects, the radius should be sufficiently large so that the change in the radius due to deployment will not result in strains greater than 45% in the stent 100 material. Conversely, in additional or alternative aspects, the radius should be sufficiently small so that the stem can be crimped sufficiently tight over a balloon.

In another aspect, the plurality of substantially straight segments 116 of each wave member 104 can further comprise a width and a thickness. In one aspect, the width and thickness are substantially equal such that the cross-section of the plurality of substantially straight segments is substantially square. In some aspects, the width of the substantially straight segment can be about 0.05 inches and, in additional or alternative aspects, the thickness of the substantially straight segment can be about 0.05 inches.

In other aspects, each adjacent pair of straight segments 116 in each wave member 104 can define a wave angle. In one aspect, the wave angle 110 can be an acute angle when the stent 100 is in a collapsed position. In another aspect, the wave angle can be about 90 degrees when the stent is in a deployed position. In light of the present disclosure, one skilled in the art will appreciate that the radial force exerted by the stent 100 can depend on both the stent diameter and the wave angle and increases as the stent is moved from the collapsed to the deployed position. It is contemplated that the stent can be adapted to have a deployed radial strength sufficient to overcome the recoil forces applied by the blood vessel and, optionally, the plaque layer, within which it is deployed.

In other aspects, the plurality of interconnected wave members 104 of each spring member can comprise from about 4 to about 12 wave members, more preferably, from about 6 to about 10 wave members and, most preferably, about 8 wave members. In other aspects, the wave amplitude 108 and wave angle 110 can determine the number of wave members of a spring member.

In one aspect, each wave member 104 of the spring member 102 can have an amplitude 108. In operation, when the stent 100 is moved from the collapsed position to the deployed position, the radii 114 of each curved segment 106 and the wave angle 110 of each wave member 104 increases, while the wave amplitude 108 decreases. Here, the wave amplitude 108 determines the moment applied on each of the plurality of curved segments and can affect the pressure at which the stent begins to expand. In one aspect, the stent 100 can be configured to expand at a relatively low pressure, i.e., between about 3 to about 5 ATM. In light of the present disclosure, one skilled in the art will appreciate the wave amplitude should not be too small and, conversely, if the wave amplitude is too high, the wave angle will not increase enough rendering the stent with insufficient radial force to withstand the recoil forces of the blood vessel and any plaque layer upon deployment. In light of the foregoing, in sonic aspects, the amplitude can be from about 1 to about 2 mm and, more preferably, can be about 1.5 mm.

In other aspects, the plurality of biasable couplings 118 have opposing ends 122, 124, wherein one end of one biasable coupling can be joined to a portion of the respective curved segment 106 of a select wave member 104 of a selected spring member 102 and the other opposing end of the one biasable coupling can be joined to a portion of the respective curved segment 106 of another select wave member 104 of an adjacent spring member. It is contemplated that the plurality of biasable couplings can be adapted to elongate upon movement of the stent 100 from the collapsed position to the deployed position to proportionally compensate for the decrease in amplitude of the plurality of wave members of the plurality of spring members 102 such that the stent maintains a substantially constant length.

In further aspects, the plurality of biasable couplings 118 can further comprise a plurality of flexible struts, each having a central axis 120 that is rotationally offset from the longitudinal axis of the stent 100. In other aspects, the plurality of flexible struts comprises a plurality of flexible S-shaped struts, each having a central axis 120 that is rotationally offset from the ring plane and, thus the longitudinal axis of the stent. In even further aspects, the plurality of flexible struts can be rotationally offset from either the longitudinal axis of the stent or the ring plane at a selected angle. In even further aspects, a selected plurality of flexible struts can be offset in a first direction by the selected angle while each adjacent plurality of flexible struts is offset in a second direction by the selected angle, where the first direction is opposite to the second direction. In operation, rotationally offsetting the central axes of the plurality of flexible struts from the longitudinal axis of the stent can improve the longitudinal flexibility of the stent as compared to a configuration where the plurality of flexible struts each have a central axis 120 aligned with the longitudinal axis of the stent 100 and, thus, increase the resistance to longitudinal bending of the stent.

In other aspects, where each of the plurality of biasable couplings 118 comprises one end joined to a portion of the respective curved segment 106 that is spaced apart from and lies to a first side of the apex 112 of a selected wave member 104 of a selected spring member 102and the opposing end of the one biasable coupling is joined to a portion of the respective curved segment 106 that is spaced apart from and to a second side of the apex 112 of another select wave member 104 of an adjacent spring member. In even further aspects, the portions of the respective curve segments to which the opposing ends 122, 124 of the biasable couplings 118 are joined can be selected to be the that portion of the respective curve segment that adjoins the straight segment. In light of the present disclosure, one skilled in the art will appreciate that one end of a selected biasable coupling (or one apex of a select wave member of a spring member) can be stretched relative to the second end of the biasable coupling (or the apex of another select wave member of an adjacent wave member) during movement of the plurality of spring members 102 from a collapsed position to a deployed position. In other aspects, it is contemplated that plurality of biasable couplings elongate to proportionally compensate for the decrease in amplitude of the plurality of wave members of the plurality of spring members such that the stent 100 maintains a substantially constant length.

In other aspect, stents of the present disclosure can be designed such that each curved segment 106 of the wave member 104 of the two adjacent spring members 102 jointed via a biasable coupling can be configured so as not to substantially change their respective circumferential positions during movement of the stent 100 between the collapsed and the deployed positions.

In alternate aspects, stents of the present disclosure can be designed such that each curved segment 106 of the wave member 104 of the two adjacent spring members 102 joined via a biasable coupling can be configured to selectively change their respective circumferential positions during movement of the stent 100 between the collapsed and the deployed positions. Here, due to the friction between the spring members and the balloon, the plurality of biasable couplings 118 can elongate while the stent 100 expands and the wave amplitude 108 decreases, thus maintaining the overall length of the stent. In even further aspects, while the spring members 102 expand at both sides of a given biasable coupling, they apply a moment on the biasable coupling operable to open the biasable coupling and assist in elongation thereof.

It is further contemplated that the stents described herein can be designed to selectively elongate along the stent axis.

In light of the present disclosure, one skilled in the art will appreciate that the number of biasable couplings 118 joining adjacent spring members 102 can impact the longitudinal flexibility of the stent 100 and, in turn, the ability of the stent to conform to the blood vessel curvature. I.e., the fewer biasable couplings joining adjacent spring members, the more longitudinally flexible the stent can be. Conversely, longitudinal stability can be beneficial during crimping, expansion and when expanded in the artery. Accordingly, the number of biasable couplings between adjacent spring members can be from about 2 to about 8 and, more preferably, from about 3 to about 4.

In yet other aspects, each of the plurality of biasable couplings 118 can have at least one of a width and a thickness thinner than that of each of the plurality of spring members 102 in order to facilitate elongation of the plurality of biasable couplings. In one exemplary embodiment where the each of the plurality of biasable couplings comprises an flexible S-shaped strut, at least one of the width and the thickness of each of the plurality of struts can be about 0.003″, the internal radius 114 of the strut can be about 0.07 mm and the distance between the internal radii of the strut can be about 0.4 mm.

In yet other aspects, the length of the connection joining the biasable couplings 118 to the spring members 102 can be minimized so as to avoid thickening of the curved segment 106 of the respective wave member 104 sufficient to materially increase the material strain during movement of the stent 100 between the collapsed and deployed positions.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

1-26. (canceled).

27. An expandable stent, comprising:
a plurality of annular spring members defining a stent having a longitudinal axis, wherein the plurality of spring members are configured to move between a collapsed position and a deployed position, wherein each spring member has a plurality of integrally connected wave members, wherein each wave member comprises at least one crest, at least one trough, two substantially straight segments disposed between a crest and a trough, and an amplitude of from about 1 mm to about 2 mm, wherein each crest and trough has an apex and a radius, and wherein the plurality of spring members are spaced along the longitudinal axis and are circumferentially oriented such that the crests of adjacent wave members are substantially identically aligned;
a first spring member of the plurality of spring members, the first spring member comprising a first selected wave member;
the second spring member of the plurality of spring members, the second spring member positioned adjacent to the first spring member along the longitudinal axis of the stent and comprising a second selected wave member;
wherein a crest of the first selected wave member is circumferentially offset from a trough of the second selected wave member,
at least one biasable S-shaped coupling positioned between the first and second selected wave members, the biasable S-shaped coupling consisting of a first elongated section extending between a first end and a first curved section, a second elongated section extending between the first curved segment and a second curved section, and a third elongated section extending between the second curved section and the second end,
wherein the first end of a selected biasable coupling is joined to the crest of the first selected wave member at a position spaced from the apex of the crest, and the second end of the selected biasable coupling is joined to the trough of the second selected
wave member at a position spaced from the apex of the trough; and
wherein the plurality of biasable S-shaped couplings are adapted to elongate upon movement of the stent from the collapsed position to the deployed position to proportionally compensate for the decrease in amplitude of the plurality of wave members of the plurality of spring members such that the stent maintains a substantially constant length.

28. The expandable stent of claim 27, wherein the amplitude is about 1.5 mm.

29. The expandable stent of claim 27, wherein the radius is from about 0.05 to about

0. 15 mm.

30. The expandable stent of claim 29, wherein the radius is about 0.1 mm.

31. The expandable stent of claim 27, wherein the plurality of wave members comprises from about 4 to about 12 wave members.

32. The expandable stent of claim 31, wherein the plurality of wave members comprises from about 6 to about 10 wave members.

33. The expandable stent of claim 32, wherein the plurality of wave members comprises about 8 wave members.

34. The expandable stent of claim 27, wherein each of the plurality of straight segments in each wave member further comprises a width and a thickness.

35. The expandable stent of claim 34, wherein the width is about 0.005 inches.

36. The expandable stent of claim 34, wherein the thickness is about 0.005 inches.

37. The expandable stent of claim 31, wherein each adjacent pair of straight segments in each wave member defines a wave angle, wherein the wave angle is an acute angle in the collapsed position.

38. The expandable stent of claim 37, wherein, in a deployed position, the wave angle can be about 90 degrees.

39. The expandable stent of claim 27, wherein each of the opposing ends of the biasable S-shaped coupling is joined to each of the adjacent spring members on the portion of the respective curved segment that adjoins a straight segment.

40. The expandable stent of claim 27, wherein the plurality of biasable S-shaped couplings can be from about 2 to about 8 biasable couplings.

41. The expandable stent of claim 40, wherein the plurality of biasable S-shaped couplings can be from about 3 to about 4 biasable couplings.

42. The expandable stent of claim 27, wherein the plurality of spring members and the plurality of biasable S-shaped couplings are formed from a stent material.

43. The expandable stent of claim 42, wherein the stent material is selected from the group comprising stainless steel and cobalt chromium alloy.

44. The expandable stent of claim 42, wherein the stent material does not develop strain greater than 45% upon movement of the stent from the collapsed position to the deployed position.

45. The expandable stent of claim 27, wherein the plurality of biasable S-shaped couplings further comprises a plurality of flexible struts having a central axis that is rotationally offset from the longitudinal axis of the stent.

46. The expandable stent of claim 27, wherein the plurality of biasable S-shaped couplings further comprises a plurality of struts, each strut having a central axis that is rotationally offset from the ring plane of an adjoining spring member at a selected angle.

47. The expandable stent of claim 46, wherein a selected plurality of struts are rotationally offset in a first direction from the ring plane of one adjoining spring member and another selected plurality of struts are rotationally offset in a second direction from the ring plane of another adjoining spring member; wherein the first direction is opposite to the second direction.
